(19) United States
(12) Patent Application Publication
(10) Pub. No.: US 2024/0110907 A1
(43) Pub. Date: Apr. 4, 2024

(54) Soil Moisture Monitoring Systems and Methods For Measuring Mutual Inductance of Area of Influence Using Radio Frequency Stimulus

(71) Applicant: FarmX Inc., Mountain View, CA (US)

(72) Inventor: William Eugene Jennings, San Jose, CA (US)

(21) Appl. No.: 17/958,253

(22) Filed: Sep. 30, 2022

(51) Int. Cl. G01N 33/24

(52) U.S. Cl. CPC G01N 33/246; G01N 2033/245

(57) ABSTRACT

Embodiments of the present disclosure are directed to device having an intelligent irrigation system comprising a soil moisture sensor further comprising a power source, a processor communicatively coupled to a memory and the power source, a GPS receiver communicatively coupled to the power source, the processor and the memory, the GPS receiver having a GPS antenna, an oscillator communicatively coupled to the power source the processor, the memory and the GPS receiver, a sensing antenna communicatively coupled to the oscillator, and the sensing antenna configured to transmit a radio frequency signal toward or into a ground surface for sensing. The power source may be supplied by a farm implement, a tractor, a local battery in the soil moisture sensor, or a replaceable battery in the soil moisture sensor. Additionally, the soil moisture sensor may be portable and can be carried by hand.

### FIELD OF THE TECHNOLOGY

Embodiments of the disclosure relate to soil moisture monitoring. Some non-limiting embodiments comprise the use of a radio frequency from a fixed point above or near a soil surface to determine moisture at varying depths.

### SUMMARY

Embodiments of the present disclosure are directed to device having an intelligent irrigation system comprising a soil moisture sensor further comprising a power source, a processor communicatively coupled to a memory and the power source, a GPS receiver communicatively coupled to the power source, the processor and the memory, the GPS receiver having a GPS antenna, an oscillator communicatively coupled to the power source the processor, the memory and the GPS receiver, a sensing antenna communicatively coupled to the oscillator, and the sensing antenna configured to transmit a radio frequency signal toward or into a ground surface for sensing. The power source may be supplied by a farm implement, a tractor, a local battery in the soil moisture sensor, or a replaceable battery in the soil moisture sensor. Additionally, the soil moisture sensor may be portable and can be carried by hand.

In various exemplary embodiments, the soil moisture sensor may be attached to a tractor or a farm implement. The tractor or farm implement may be manually or autonomously operated. All data may be collected, processed and stored within the soil moisture sensor. Additionally, a network may be included and an integrated radio, cellular connection, satellite connection, or an industrial/scientific/medical (ISM) radio is used to communicate with the network. The ISM radio may use a LoRaWAN protocol, having a LoRa gateway. A cloud resource may include a neural network. The oscillator, in some exemplary embodiments, may be a Hartley oscillator configured to operate at 60/120 primary and secondary frequencies.

Exemplary methods of using an intelligent irrigation system may include transmitting a radio frequency signal toward or into a ground surface, the radio frequency signal resulting in an indication of a magnitude of moisture, measuring the indication of a magnitude of moisture at a depth of 24 inches or more and transmitting the indication of the magnitude of moisture to the soil moisture sensor. Further methods may include increasing the depth measured by increasing voltage to an oscillator, decreasing the depth measured by decreasing voltage to an oscillator, measuring moisture at selective depths by taking readings at lower to higher voltages, and increasing an area of soil being measured. Additionally, slicing moisture indications by section may be performed.

A machine learning method in exemplary embodiments may include employing an intelligent irrigation system comprising using inductive sensing to determine a soil's magnetic permeability, measuring a frequency soil moisture during a known short measurement period and transmitting a count to a cloud resource or transmitting to a local memory until a network is available to connect to the cloud resource. Methods may include converting the count to a volumetric water content based on soil type and calibration. A soil percolation model may be developed to track in-field moisture, and methods may also include dynamically adjusting soil and site-specific calibration for increased accuracy and geo-tagging data when sampled. Additionally, correlating time-sequence data from multiple readings over multiple visits may be performed and logging longitude, latitude, and altitude for each reading may occur.

### BRIEF DESCRIPTION OF THE DRAWINGS

While this technology is susceptible of embodiment in many different forms, there is illustrated in the drawings, and will herein be described in detail, several specific embodiments with the understanding that the present disclosure is to be considered as an exemplification of the principles of the technology and is not intended to limit the technology to the embodiments illustrated.

It will be understood that like or analogous elements and/or components, referred to herein, may be identified throughout the drawings with like reference characters. It is further understood that several of the figures are merely schematic representations of the present technology. As such, some of the components may be distorted from their actual scale for pictorial clarity.

FIG. 1 shows an exemplary intelligent irrigation system.

FIG. 2 shows an exemplary method of using an intelligent irrigation system.

FIG. 3 shows an exemplary machine learning method employing an intelligent irrigation system.

FIG. 4 shows another exemplary method of using an intelligent irrigation system.

FIG. 5 shows an exemplary deep neural network.

### DETAILED DESCRIPTION OF EXEMPLARY EMBODIMENTS

The exemplary embodiments provided herein are for use in irrigation agriculture, particularly permanent crops. More specifically, the exemplary embodiments comprise a system and method for accurately measuring the volumetric water content of soil using a cost-effective apparatus.

For context, of the principal ways that farmers manage their irrigation is with soil moisture sensors. In brief, plants uptake their water from the soil through their root structure. Their ability to do so is impacted by the amount of water in the soil, the soil tension, and the suction force needed by the roots to absorb water.

Other sensor designs have emulated the root of a plant and measure the water tension in the soil. These are known as tensiometers. There are sensors that look like forks that have two parallel probes about an inch apart and measure the capacitance between the probes. This is calibrated to the amount of moisture, and the calibration is dependent on the soil type that the probe measures. There are probes that use gymsum blocks that absorb water, and as these sensors absorb water their resistance reduces changes in a manner that is correlated to the amount of water absorbed. These probes have a short lifespan, as the process of absorbing moisture causes them to deteriorate. Further, their installation takes weeks before readings are meaningful.

Liquid sensing using radio frequency techniques is a known mechanism in industrial settings. One such reference would be: Liquid Sensing at Radio Frequencies, Complex impedance measurement of liquid samples as a function of frequency, Microwave Journal, Thomas J. Warnagiris, Sep. 1, 2000.

Until now, complete soil moisture sensors have not been produced in a manner that are effective for agriculture, using radio frequency sensing, that are low power, easy to install, and can measure large volumes of soil accurately.

The systems and methods provided herein include a soil moisture sensor that is highly accurate, measures a larger volume of influence than other sensors, easy to install, and operates without soil specific calibration requirements.

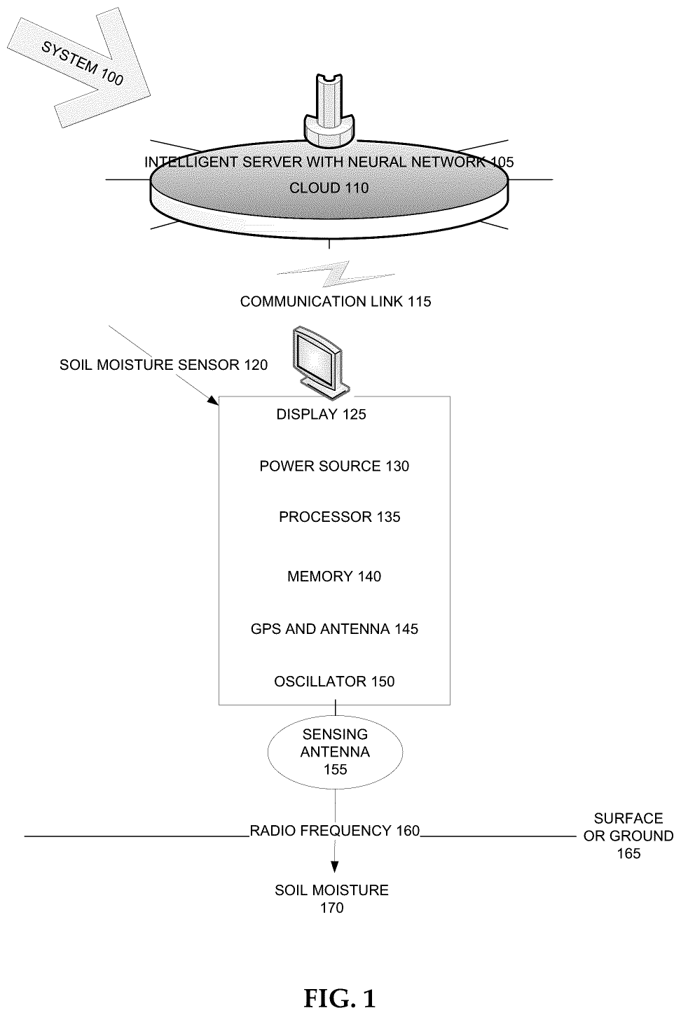

FIG. 1 shows an exemplary intelligent irrigation system.

Shown in system 100 are intelligent server with neural network 105, cloud 110, communication link 115, soil moisture sensor 120, display 125, power source 130, processor 135, memory 140, geo-positioning system (“GPS”) and antenna 145, oscillator 150, sensing antenna 155, radio frequency 160, surface or ground 165, and soil moisture 170.

Embodiments of the present disclosure are directed to device having an intelligent irrigation system comprising a soil moisture sensor further comprising a power source, a processor communicatively coupled to a memory and the power source, a GPS receiver communicatively coupled to the power source, the processor and the memory, the GPS receiver having a GPS antenna, an oscillator communicatively coupled to the power source the processor, the memory and the GPS receiver, a sensing antenna communicatively coupled to the oscillator, and the sensing antenna configured to transmit a radio frequency signal toward or into a ground surface for sensing. The power source may be supplied by a farm implement, a tractor, a local battery in the soil moisture sensor, or a replaceable battery in the soil moisture sensor. Additionally, the soil moisture sensor may be portable and can be carried by hand.

In various exemplary embodiments, the soil moisture sensor may be attached to a tractor or a farm implement. The tractor or farm implement may be manually or autonomously operated. All data may be collected, processed and stored within the soil moisture sensor. Additionally, a network may be included and an integrated radio, cellular connection, satellite connection, or an industrial/scientific/medical (ISM) radio is used to communicate with the network. The ISM radio may use a LoRaWAN protocol, having a LoRa gateway.

A cloud resource may include a neural network. The oscillator, in some exemplary embodiments, may be a Hartley oscillator configured to operate at 60/120 primary and secondary frequencies.

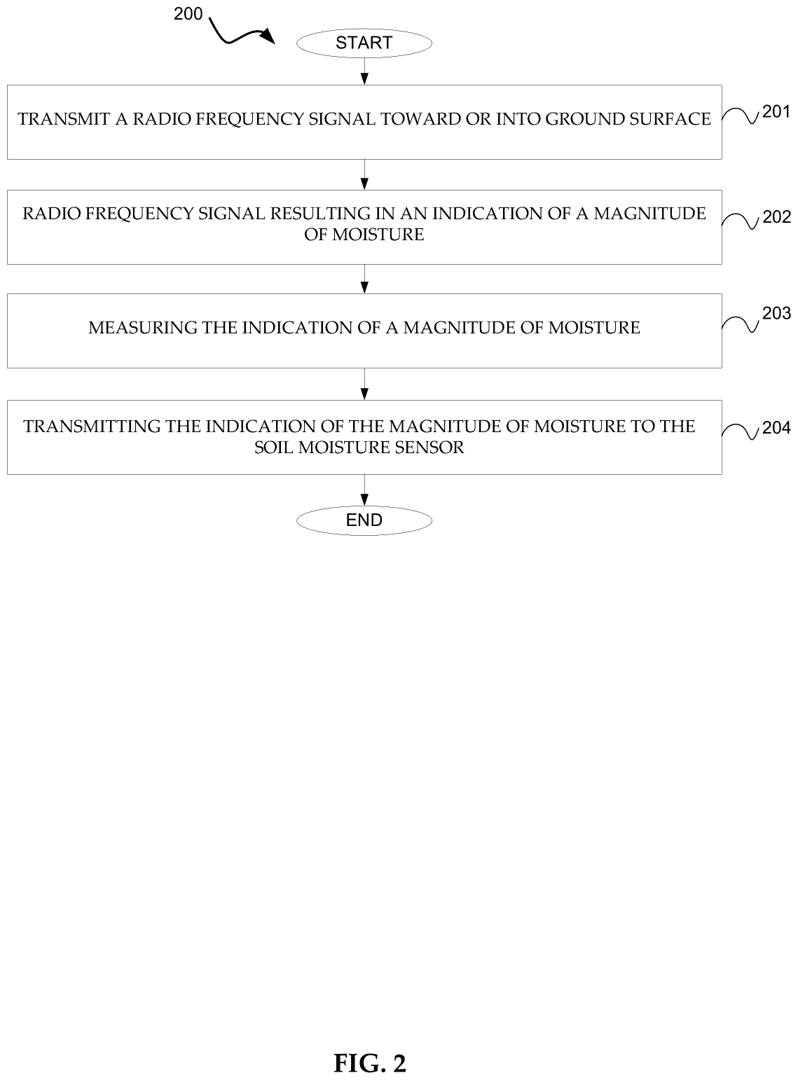

FIG. 2 shows an exemplary method of using an intelligent irrigation system. Shown in method 200 are:

Step 201: transmit a radio frequency signal toward or into a ground surface.

Step 202: radio frequency signal resulting in an indication of a magnitude of moisture.

Step 203: measuring the indication of the magnitude of moisture.

Step 204: transmitting the indication of the magnitude of moisture to the soil moisture sensor.

Exemplary methods of using an intelligent irrigation system may include transmitting a radio frequency signal toward or into a ground surface, the radio frequency signal resulting in an indication of a magnitude of moisture, measuring the indication of a magnitude of moisture at a depth of 24 inches or more and transmitting the indication of the magnitude of moisture to the soil moisture sensor. Further methods may include increasing the depth measured by increasing voltage to an oscillator, decreasing the depth measured by decreasing voltage to an oscillator, measuring moisture at selective depths by taking readings at lower to higher voltages, and increasing an area of soil being measured. Additionally, slicing moisture indications by section may be performed.

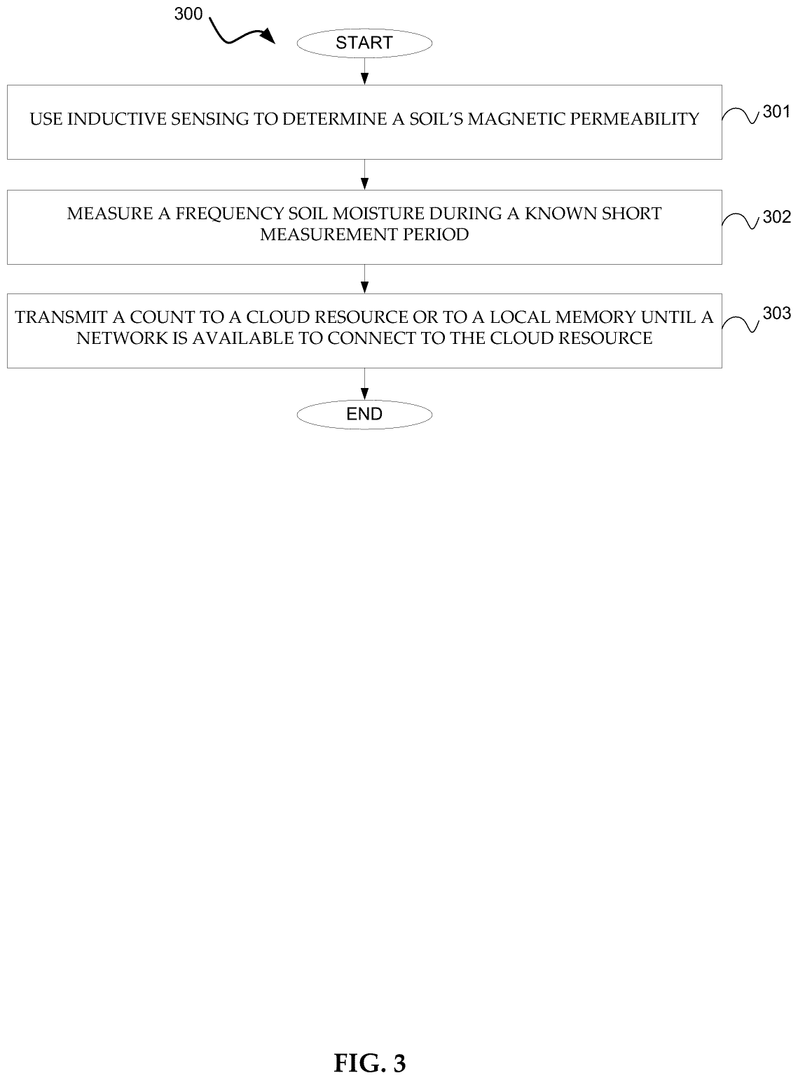

FIG. 3 shows an exemplary machine learning method employing an intelligent irrigation system.

Shown in method 300 are:

Step 301: use inductive sensing to determine a soil's magnetic permeability.

Step 302: measure a frequency soil moisture during a known short measurement period.

Step 303: transmit a count to a cloud resource or to a local memory until a network is available to connect to the cloud resource.

A machine learning method in exemplary embodiments may include employing an intelligent irrigation system comprising using inductive sensing to determine a soil's magnetic permeability, measuring a frequency soil moisture during a known short measurement period and transmitting a count to a cloud resource or transmitting to a local memory until a network is available to connect to the cloud resource. Methods may include converting the count to a volumetric water content based on soil type and calibration. A soil percolation model may be developed to track in-field moisture, and methods may also include dynamically adjusting soil and site-specific calibration for increased accuracy and geo-tagging data when sampled. Additionally, correlating time-sequence data from multiple readings over multiple visits may be performed and logging longitude, latitude, and altitude for each reading may occur.

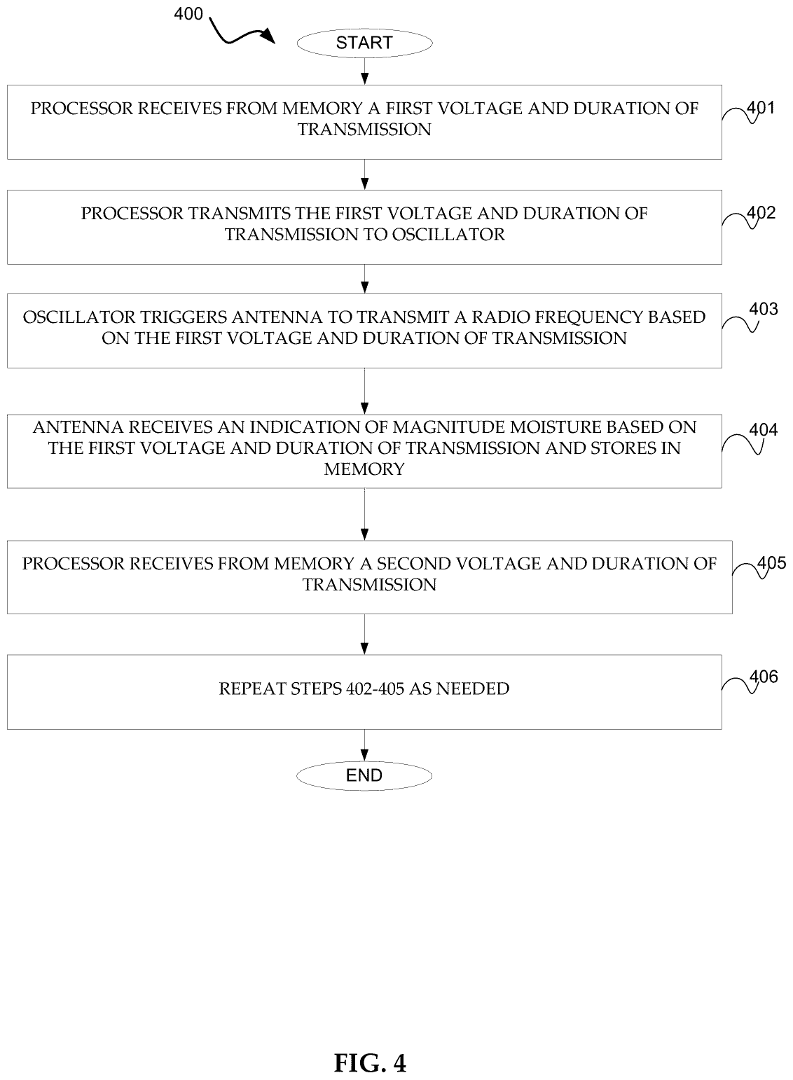

FIG. 4 shows another exemplary method of using an intelligent irrigation system.

Method 400 includes:

Step 401: the processor receives from memory a first voltage and duration of transmission.

Step 402: the processor transmits the first voltage and duration of transmission to oscillator.

Step 403: the oscillator triggers antenna to transmit a radio frequency based on the first voltage and duration of transmission.

Step 404: the antenna receives an indication of the magnitude of moisture based on the first voltage and duration of transmission and stores in the memory.

Step 405: the processor receives from the memory a second voltage and duration of transmission.

Step 406: steps 402-405 are repeated as necessary to complete the analysis of soil moisture.

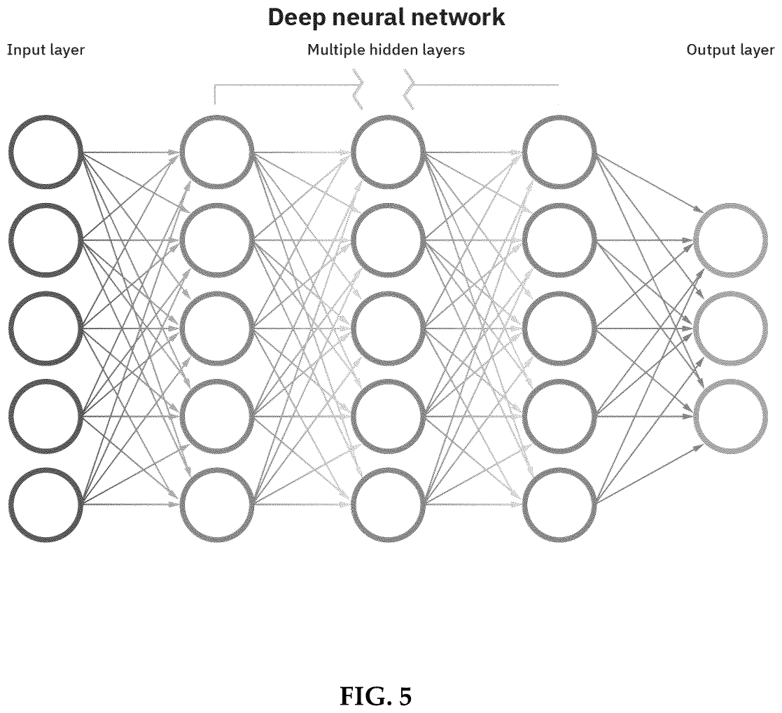

FIG. 5 shows an exemplary deep neural network.

Neural networks, also known as artificial neural networks (ANNs) or simulated neural networks (SNNs), are a subset of machine learning and are at the heart of deep learning algorithms. Their name and structure are inspired by the human brain, mimicking the way that biological neurons signal to one another. Artificial neural networks (ANNs) are comprised of a node layers, containing an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed along to the next layer of the network.

Neural networks rely on training data to learn and improve their accuracy over time. However, once these learning algorithms are fine-tuned for accuracy, they are powerful tools in computer science and artificial intelligence, allowing one to classify and cluster data at a high velocity. Tasks in speech recognition or image recognition can take minutes versus hours when compared to the manual identification by human experts. One of the most well-known neural networks is Google's search algorithm.

In some exemplary embodiments, one should view each individual node as its own linear regression model, composed of input data, weights, a bias (or threshold), and an output. Once an input layer is determined, weights are assigned. These weights help determine the importance of any given variable, with larger ones contributing more significantly to the output compared to other inputs. All inputs are then multiplied by their respective weights and then summed. Afterward, the output is passed through an activation function, which determines the output. If that output exceeds a given threshold, it “fires” (or activates) the node, passing data to the next layer in the network. This results in the output of one node becoming in the input of the next node. This process of passing data from one layer to the next layer defines this neural network as a feedforward network. Larger weights signify that particular variables are of greater importance to the decision or outcome.

Most deep neural networks are feedforward, meaning they flow in one direction only, from input to output. However, one can also train a model through backpropagation; that is, move in the opposite direction from output to input. Backpropagation allows one to calculate and attribute the error associated with each neuron, allowing one to adjust and fit the parameters of the model(s) appropriately.

In machine learning, backpropagation is an algorithm for training feedforward neural networks. Generalizations of backpropagation exist for other artificial neural networks (ANNs), and for functions generally. These classes of algorithms are all referred to generically as “backpropagation”. In fitting a neural network, backpropagation computes the gradient of the loss function with respect to the weights of the network for a single input-output example, and does so efficiently, unlike a naive direct computation of the gradient with respect to each weight individually. This efficiency makes it feasible to use gradient methods for training multilayer networks, updating weights to minimize loss; gradient descent, or variants such as stochastic gradient descent, are commonly used. The backpropagation algorithm works by computing the gradient of the loss function with respect to each weight by the chain rule, computing the gradient one layer at a time, iterating backward from the last layer to avoid redundant calculations of intermediate terms in the chain rule; this is an example of dynamic programming. The term backpropagation strictly refers only to the algorithm for computing the gradient, not how the gradient is used; however, the term is often used loosely to refer to the entire learning algorithm, including how the gradient is used, such as by stochastic gradient descent. Backpropagation generalizes the gradient computation in the delta rule, which is the single-layer version of backpropagation, and is in turn generalized by automatic differentiation, where backpropagation is a special case of reverse accumulation (or “reverse mode”).

With respect to FIG. 5, according to exemplary embodiments, the system produces an output, which in turn produces an outcome, which in turn produces an input. In some embodiments, the output may become the input.

Here, the deep neural network may have a wealth of collected information, including soil moisture measurements over time, crop type, GPS readings, weather, soil depth, rain fall, energy costs, ripening and harvest times. In turn, the deep neural network may generate output information to formulate a strategy. Application of the strategy may result in an outcome that may be fed back into the deep neural network for formulating an improved strategy, which may lead to increased profits along with optimization of natural and non-renewable resources required to run an operation.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description, but is not intended to be exhaustive or limited to the present technology in the form disclosed. Many modifications and variations will be apparent to those of ordinary skill in the art without departing from the scope and spirit of the present technology. Exemplary embodiments were chosen and described in order to best explain the principles of the present technology and its practical application, and to enable others of ordinary skill in the art to understand the present technology for various embodiments with various modifications as are suited to the particular use contemplated.

Aspects of the present technology are described above with reference to flowchart illustrations and/or block diagrams of methods, apparatus (systems) and computer program products according to embodiments of the present technology. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

In the following description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It is noted at the outset that the terms “coupled,” “connected”, “connecting,” “electrically connected,” etc., are used interchangeably herein to generally refer to the condition of being electrically/electronically connected. Similarly, a first entity is considered to be in “communication” with a second entity (or entities) when the first entity electrically sends and/or receives (whether through wireline or wireless means) information signals (whether containing data information or non-data/control information) to the second entity regardless of the type (analog or digital) of those signals. It is further noted that various figures (including component diagrams) shown and discussed herein are for illustrative purpose only, and are not drawn to scale.

If any disclosures are incorporated herein by reference and such incorporated disclosures conflict in part and/or in whole with the present disclosure, then to the extent of conflict, and/or broader disclosure, and/or broader definition of terms, the present disclosure controls. If such incorporated disclosures conflict in part and/or in whole with one another, then to the extent of conflict, the later-dated disclosure controls.

The terminology used herein can imply direct or indirect, full or partial, temporary or permanent, immediate or delayed, synchronous or asynchronous, action or inaction. For example, when an element is referred to as being “on,” “connected” or “coupled” to another element, then the element can be directly on, connected or coupled to the other element and/or intervening elements may be present, including indirect and/or direct variants. In contrast, when an element is referred to as being “directly connected” or “directly coupled” to another element, there are no intervening elements present.

Although the terms first, second, etc. may be used herein to describe various elements, components, regions, layers and/or sections, these elements, components, regions, layers and/or sections should not necessarily be limited by such terms. These terms are only used to distinguish one element, component, region, layer or section from another element, component, region, layer or section. Thus, a first element, component, region, layer or section discussed below could be termed a second element, component, region, layer or section without departing from the teachings of the present disclosure.

The terminology used herein is for the purpose of describing particular embodiments only and is not intended to be necessarily limiting of the disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence or addition of one or more other features, integers, steps, operations, elements, components, and/or groups thereof.

Example embodiments of the present disclosure are described herein with reference to illustrations of idealized embodiments (and intermediate structures) of the present disclosure. As such, variations from the shapes of the illustrations as a result, for example, of manufacturing techniques and/or tolerances, are to be expected. Thus, the example embodiments of the present disclosure should not be construed as necessarily limited to the particular shapes of regions illustrated herein, but are to include deviations in shapes that result, for example, from manufacturing.

Any and/or all elements, as disclosed herein, can be formed from a same, structurally continuous piece, such as being unitary, and/or be separately manufactured and/or connected, such as being an assembly and/or modules. Any and/or all elements, as disclosed herein, can be manufactured via any manufacturing processes, whether additive manufacturing, subtractive manufacturing and/or other any other types of manufacturing. For example, some manufacturing processes include three dimensional (3D) printing, laser cutting, computer numerical control (CNC) routing, milling, pressing, stamping, vacuum forming, hydroforming, injection molding, lithography and/or others.

Any and/or all elements, as disclosed herein, can include, whether partially and/or fully, a solid, including a metal, a mineral, a ceramic, an amorphous solid, such as glass, a glass ceramic, an organic solid, such as wood and/or a polymer, such as rubber, a composite material, a semiconductor, a nano-material, a biomaterial and/or any combinations thereof. Any and/or all elements, as disclosed herein, can include, whether partially and/or fully, a coating, including an informational coating, such as ink, an adhesive coating, a melt-adhesive coating, such as vacuum seal and/or heat seal, a release coating, such as tape liner, a low surface energy coating, an optical coating, such as for tint, color, hue, saturation, tone, shade, transparency, translucency, non-transparency, luminescence, anti-reflection and/or holographic, a photo-sensitive coating, an electronic and/or thermal property coating, such as for passivity, insulation, resistance or conduction, a magnetic coating, a water-resistant and/or waterproof coating, a scent coating and/or any combinations thereof.

Unless otherwise defined, all terms (including technical and scientific terms) used herein have the same meaning as commonly understood by one of ordinary skill in the art to which this disclosure belongs. The terms, such as those defined in commonly used dictionaries, should be interpreted as having a meaning that is consistent with their meaning in the context of the relevant art and should not be interpreted in an idealized and/or overly formal sense unless expressly so defined herein.

Furthermore, relative terms such as “below,” “lower,” “above,” and “upper” may be used herein to describe one element's relationship to another element as illustrated in the accompanying drawings. Such relative terms are intended to encompass different orientations of illustrated technologies in addition to the orientation depicted in the accompanying drawings. For example, if a device in the accompanying drawings is turned over, then the elements described as being on the “lower” side of other elements would then be oriented on “upper” sides of the other elements. Similarly, if the device in one of the figures is turned over, elements described as “below” or “beneath” other elements would then be oriented “above” the other elements. Therefore, the example terms “below” and “lower” can, therefore, encompass both an orientation of above and below.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments. cm What is claimed is:

1. An intelligent irrigation system comprising:
a soil moisture sensor further comprising:
a power source;
a processor communicatively coupled to a memory and the power source;
a GPS receiver communicatively coupled to the power source, the processor and the memory, the GPS receiver having a GPS antenna;
an oscillator communicatively coupled to the power source the processor, the memory and the GPS receiver;
a sensing antenna communicatively coupled to the oscillator;
the sensing antenna configured to transmit a radio frequency signal toward or into a ground surface for sensing.

2. The soil moisture sensor of claim 1, wherein the power source is supplied by a farm implement, a tractor, a local battery in the soil moisture sensor, or a replaceable battery in the soil moisture sensor.

3. The soil moisture sensor of claim 1, wherein the soil moisture sensor is portable and can be carried by hand.

4. The soil moisture sensor of claim 1, further comprising the soil moisture sensor attached to a tractor or a farm implement.

5. The soil moisture sensor of claim 4, wherein the tractor or farm implement is manually or autonomously operated.

6. The soil moisture sensor of claim 1, wherein all data is collected, processed and stored within the soil moisture sensor.

7. The system of claim 1, further comprising a network.

8. The system of claim 7, wherein an integrated radio, cellular connection, satellite connection, or an industrial/scientific/medical (ISM) radio is used to communicate with the network.

9. The system of claim 8, wherein the ISM radio uses a LoRaWAN protocol, having a LoRa gateway.

10. The system of claim 1, further comprising a cloud resource.

11. The system of claim 10, further comprising a neural network.

12. The soil moisture sensor of claim 1, wherein the oscillator is a Hartley oscillator configured to operate at 60/120 primary and secondary frequencies.

13. A method of using an intelligent irrigation system, the method comprising:
transmitting a radio frequency signal toward or into a ground surface, the radio frequency signal resulting in an indication of a magnitude of moisture;
measuring the indication of a magnitude of moisture at a depth of 24 inches or more; and
transmitting the indication of the magnitude of moisture to the soil moisture sensor.

14. The method of claim 13, further comprising: increasing the depth measured by increasing voltage to an oscillator.

15. The method of claim 13, further comprising: decreasing the depth measured by decreasing voltage to an oscillator.

16. The method of claim 13, further comprising: measuring moisture at selective depths by taking readings at lower to higher voltages.

17. The method of claim 13, further comprising: increasing an area of soil being measured.

18. The method of claim 13, further comprising: slicing moisture indications by section.

19. A machine learning method employing an intelligent irrigation system,
the method comprising:
using inductive sensing to determine a soil's magnetic permeability;
measuring a frequency soil moisture during a known short measurement period; and
transmitting a count to a cloud resource or transmitting to a local memory until a network is available to connect to the cloud resource.

20. The machine learning method of claim 19, further comprising: converting the count to a volumetric water content based on soil type and calibration.

21. The machine learning method of claim 20, further comprising: developing a soil percolation model to track in-field moisture.

22. The machine learning method of claim 21, further comprising:
dynamically adjusting soil and site-specific calibration for increased accuracy.

23. The machine learning method of claim 19, further comprising: geo-tagging data when sampled.

24. The machine learning method of claim 23, further comprising: correlating time-sequence data from multiple readings over multiple visits.

25. The machine learning method of claim 24, further comprising: logging longitude, latitude, and altitude for each reading.
